(19) United States
(12) Patent Application Publication
(10) Pub. No.: US 2018/0249953 A1
(43) Pub. Date: Sep. 6, 2018

(54) SYSTEMS AND METHODS FOR SURGICAL TRACKING AND VISUALIZATION OF HIDDEN ANATOMICAL FEATURES

(71) Applicant: The Charles Stark Draper Laboratory, Inc., Cambridge, MA (US)

(72) Inventors: Andrew A. Berlin, Lexington, MA (US); Philip D. Parks, II, Wayland, MA (US)

(21) Appl. No.: 15/909,282

(22) Filed: Mar. 1, 2018

Related U.S. Application Data
(60) Provisional application No. 62/466,339, filed on Mar. 2, 2017.

(51) Int. Cl. A61B 5/00; A61B 5/04; A61B 5/0488; A61B 34/20; A61B 34/10; G06T 7/246; G06T 7/73

(52) U.S. Cl. CPC A61B 5/4893; A61B 5/04001; A61B 5/7445; A61B 5/725; A61B 34/30; A61B 34/20; A61B 34/10; G06T 7/246; G06T 7/73; A61B 5/0488

(57) ABSTRACT

This invention provides systems and methods for using spatially localized sensor data to construct a multi-dimensional map of the location of key anatomical features, as well as systems and methods for utilizing this map to present location-based information to the surgeon and/or to an automated surgical navigation system. The location map is updated during surgery, so as to retain accuracy even in the presence of muscle-induced motion or deformation of the anatomy, as well as tissue location changes induced by translations or deformations induced by surgical dissection, surgical instrument contact, or biologically-induced tissue motion associated with activities such as respiration, anesthesia-induced gag reflex, blood flow/pulsation, and/or larger-scale changes in patient posture/positioning.

### RELATED APPLICATION

This application claims the benefit of co-pending U.S. Provisional Application Ser. No. 62/466,339, entitled SYSTEMS AND METHODS FOR SURGICAL TRACKING AND VISUALIZATION OF HIDDEN ANATOMICAL FEATURES, filed Mar. 2, 2017, the teachings of which are expressly incorporated herein by reference.

### FIELD OF THE INVENTION

This invention relates to surgical procedures and more particularly to systems and methods for locating hidden anatomical features during surgery, such as nerves.

### BACKGROUND OF THE INVENTION

One of the most significant challenges in surgery is protecting hidden anatomical features such as nerves and arteries. Oftentimes these features are visually indistinguishable from other types of tissues, or are embedded within tissues that obscure their specific location. Existing techniques for image-guided surgery (which can be manually or robotically performed) attempt to address this issue by optically observing and tracking the visible features of a structure of interest (such as the liver) and fitting a predetermined anatomical model of hidden features such as the vasculature to that structure. For example, presenting a conventional textbook vasculature model as an overlay on top of a video representation of the organ being operated upon. However, in many situations this approach is not effective because the actual anatomy differs from that of the textbook, which can be termed “aberrant anatomy”. For example, in situations such as nerve-sparing prostatectomy surgery, the location of key nerves varies significantly from the ‘typical’ pattern in about 50% of patients. This makes it highly advantageous to detect, track, and represent the actual structure of the anatomy of the patient being operated upon. In many cases these structures cannot be visualized pre-operatively using medical imaging, either due to insufficient contrast relative to the surrounding tissue or due to size (i.e. the structures are too small to see at the resolution of an MRI scanner).

Commercially available products and associated techniques for tracking nerves, such as the ProPep® Nerve Monitoring System by ProPep Surgical of Austin, Tex. and/or the NIM nerve monitoring system by Medtronic of Minneapolis, Minn., typically involve the use of stimulation probes that excite the nerve at a specific location during surgery, together with EMG electrodes that detect the response of the muscle that the nerve innervates. Electromyography (EMG) is a diagnostic procedure typically employed to assess the health of muscles and the nerve cells that control them (motor neurons). Motor neurons transmit electrical signals under stimulation that cause muscles to contract. An EMG translates these signals into graphs, sounds or numerical values that a practitioner can interpret. In the current state of the art, stimulation of the nerve is performed at a single location, controlled by the surgeon using either a handheld probe or a robotically actuated probe. The resulting muscle stimulation is detected via EMG, with the EMG waveform displayed on a monitor in the operating room and in some cases accompanied by an audio or visual notification that a nerve has been detected at the present location of the probe. However, the information generated by this approach is based upon a single reference in time and space. It is thereafter left to the surgeon to form a mental map of the probable nerve routing based on observation of the EMG responses achieved at each of multiple stimulation sites that may be performed during the surgical procedure. Even if the surgeon can form an adequate mental map of the site based upon the stimulation procedure, it is recognized that organs and other bodily tissue tend to move during surgery—sometimes as a result of the applied stimulation itself. Thus, even the most accurate mental map can be compromised by movement, which can deform the organ in ways that make it difficult to find a previously localized nerve or other hidden structure.

### SUMMARY OF THE INVENTION

This invention overcomes disadvantages of the prior art by providing systems and methods for using spatially localized sensor data to construct a multi-dimensional map of the location of key anatomical features, as well as systems and methods for utilizing this map to present location-based information to the surgeon and/or to an automated surgical navigation system. The location map is updated during surgery, so as to retain accuracy even in the presence of muscle-induced motion or deformation of the anatomy, as well as tissue location changes induced by translations or deformations induced by surgical dissection, surgical instrument contact, or biologically-induced tissue motion associated with activities such as respiration, anesthesia-induced gag reflex, blood flow/pulsation, and/or larger-scale changes in patient posture/positioning.

A system and method for spatial mapping of hidden features in a tissue is provided. A source of sensed point data is established with respect to a coordinate system relative to a region of tissue containing the hidden features. A mapping module builds data related to hidden feature locations in the coordinate system based upon at least one of (a) sensed motion in the tissue, (b) stored anatomical models, and (c) stored information related to the tissue. A data storage module stores the hidden feature locations to either locate or avoid the hidden features during a surgical procedure. Illustratively, the hidden features can comprise one or more nerve paths in the tissue. The sensed point data is determined using a nerve stimulator in combination with a sensor that measures response to stimulation—for example an EMG device. The mapping module includes an interpolation process that fills in nerve path regions between the sensed point data. The mapping module can be updated in real-time based on sensor data and/or can be augmented by pre-operative imaging data of the tissue. Illustratively, the nerve path is displayed to a user and/or utilized by an automated surgical guidance system, and the display can include at least one of a VR and AR display. In embodiments, the mapping module can be provided with images of the tissue in a plurality of positions based upon motion, in which the images include identified features of interest, and the mapping module includes an estimation process that is arranged to estimate locations of the features of interest in each of the plurality of positions. These estimated locations can fill in a position in which one or more of the features of interest are temporarily blocked from view by an obscuration. Additionally, the features of interest can be established in the texture of the tissue based upon variations in at least one of color, edges and grayscale level, and the obscuration can comprise at least one of glare, shadow and instrument occlusion. Illustratively, the estimation process can employ a stored model of the dynamics of the motion to determine a location of the one or more temporarily blocked features of interest based upon locations of the one or more features of interest when unblocked from view. The estimation process can include a Kalman filter process. The stored information can include one or more unexplored regions of the tissue that are free of sensed point data. Indications of the unexplored region(s) can be provided to the user in a variety of ways (e.g. a display overlay) that assist in guiding the user to probe such regions with the sensing arrangement. Information related to the unexplored regions can be provided to a robotic sensing arrangement to guide further sensing operations in the unexplored regions. Once probed/sensed, the region is mapped (e.g. as a no go or safe region, based on the presence or absence of hidden features, respectively). Generally, the hidden feature locations can define a multi-dimensional (i.e. at least two-dimensional or three-dimensional in various embodiments) map of hidden feature locations. This multi-dimensional map can be incorporated into the coordinate system relative to the region of tissue. In this manner multiple sensed data points can be observed simultaneously relative to the tissue.

### BRIEF DESCRIPTION OF THE DRAWINGS

The invention description below refers to the accompanying drawings, of which:

FIG. 1 is a diagram of an exemplary surgical arrangement 100 including various instruments, interfaces, an EMG (or other nerve-stimulation/recording) implementation, and a mapping/location process(or), according to an illustrative embodiment;

FIG. 2 is a flow diagram showing a procedure for tracking motion of points established on a tissue or organ and developing a deformation model therefor;

FIG. 3 is a flow diagram showing s procedure for using a database of nerve paths to determine regions in which nerves are present on tissue and avoid surgical procedures applied to such regions;

FIG. 4 is a flow diagram showing the construction of a nerve path on the tissue based upon identified points and storage of the path in a database as part of an overall map of the tissue region;

FIG. 5 is a flow diagram showing a basic procedure for locating points on the tissue that are in proximity to nerves and recording points in a database for use in associated procedures herein;

FIG. 6 is a diagram of an exemplary tissue region containing a textbook nerve path and actual nerve path established by the procedures herein; and

FIG. 7 is an exemplary screen display of a region of tissue undergoing stimulation by a robot-mounted probe and the resulting response, which allows mapping of a nerve path within the tissue by the system and method.

### DETAILED DESCRIPTION

### I. System Overview

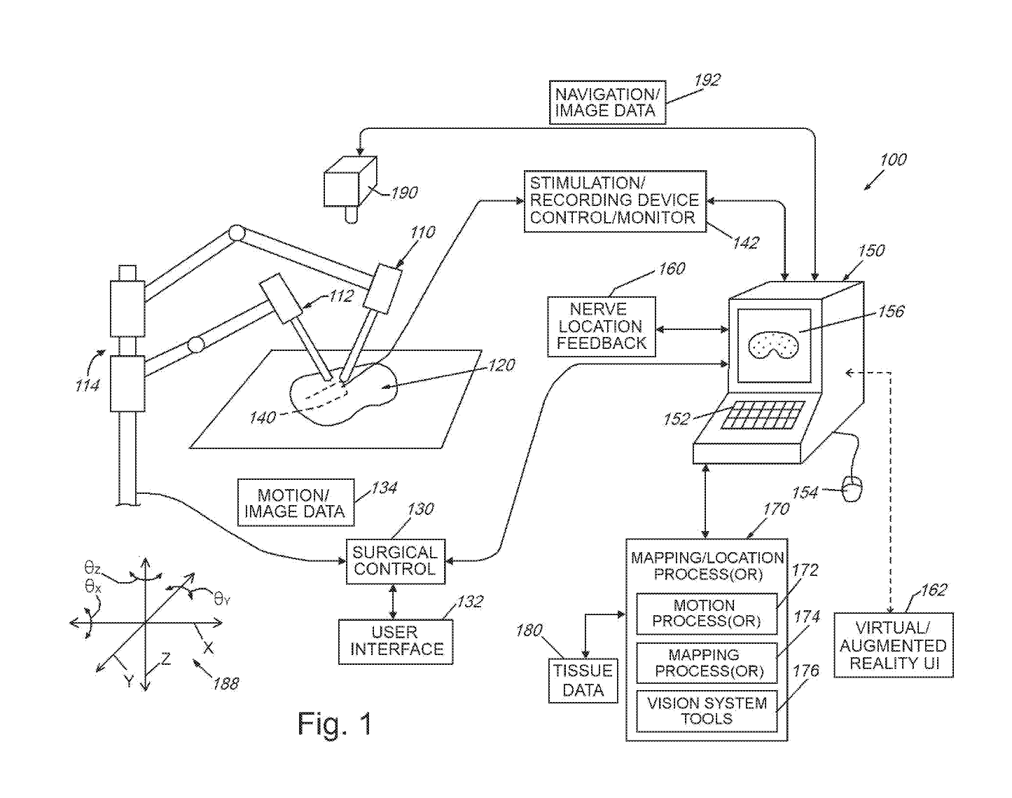

Reference is made to FIG. 1 which shows an exemplary surgical arrangement 100, that facilitates an associated surgical procedure. The arrangement and procedure, in this non-limiting example, is implemented by robotic (e.g. minimally invasive/laparoscopic) instruments 110, 112 that are mounted on a stand 114 that can be manually articulated or moved via robotic actuators and controllers along a plurality of degrees of freedom. In alternate arrangements, it is contemplated that surgery can be performed via open-cut or similar techniques using freehand-operated instruments. The instruments are shown inserted through incisions into a patient's body at an appropriate location/site (e.g. abdomen, groin, throat, head, etc.) 120, wherein they selectively engage one or more organs or other anatomical structures (e.g. muscles, vasculature, glands, ducts, etc.).

The instruments 110, 112 can include an appropriate video camera assembly with one or more image sensors that create a two-dimensional (2D) or three-dimensional (3D) still image(s) or moving image(s) of the site 120. The image sensors can include magnifying optics where appropriate and can focus upon the operational field of instrument's distally mounted tool(s). These tools can include forceps, scissors, cautery tips, scalpels, syringes, suction tips, etc., in a manner clear to those of skill. The control of the instruments, as well as a visual display can be provided by interface devices 130 and 132, respectively, based upon image/motion/location data 134 transmitted from the instruments 110, 112 and corresponding robotic components (if any).

Illustratively, one or more probes 140 are shown inserted into the site, where they engage an affected tissue/organ at locations that are meant to stimulate and record stimulus responses. The probes 140 are interconnected to a control unit and monitor 142 of a stimulation and recording device, which can be a commercially available or customized EMG device as described generally above. This device provides stimulus via the probes at different locations within the tissue, and measures the muscular response thereto. While an EMG-based device 142 is shown, this device can substituted with or supplemented with other types of simulation/recording devices that sense or detect the presence/absence of nerves within the tissue—for example an MM-based imager, and the description herein should be taken broadly to include a variety of nerve-location devices and methodologies. Likewise, it is contemplated that sensing can be accomplished using magnetic-based sensing devices, such that once excited, a nerve response can be detected via such magnetic sensors. Recent studies indicate that magnetic fields can potentially be employed to detect presence of a nerve once locally stimulated electrically, as their electrical potential affects local magnetic fields in accordance with physical laws.

By way of non-limiting example, the stimulation/recording device 142 device (EMG or similar mechanism) transmits data to a computing device 150, which can be implemented as a customized data processing device or as a general purpose computing device, such as a desktop PC, server, laptop, tablet, smartphone and/or networked “cloud” computing arrangement. The computing device 150 includes appropriate network and device interfaces (e.g. USB, Ethernet, WiFi, Bluetooth®, etc.) to support data acquisition from external devices, such as the stimulation/recording device 142 and surgical control/interface devices 130, 132. These network and data interfaces also support data transmission/receipt to/from external networks (e.g. the Internet) and devices, which can include various types of nerve location and nerve mapping feedback devices 160, as described below. The computing device 150 can include various user interface (UI) components, such as a keyboard 152, mouse 154 and/or display/touchscreen 156 that can be implemented in a manner clear to those of skill. The computing device 150 can also be arranged to interface with, and/or control, visualization devices, such as virtual reality (VR) and augmented reality (AR) user interfaces (UIs) 162. These devices can be used to assist in visualizing and/or guiding a surgeon (e.g. in real-time) using overlays of nerve structures on an actual or synthetic image of the tissue/organ being operated upon.

The computing device 150 includes a mapping and location process(or) 170 according to embodiments herein, which can be implemented in hardware, software, or a combination thereof. The mapping/location processor 170 receives data from the stimulation/recording device(s) 142 and from the surgical control and interface 130, 132 devices, and uses this information, in combination with additional data 180 on the characteristics of the subject tissue/organ. As described further below, this data can include textbook nerve path locations, the manner in which tissue shifts during normal motion and the shape such tissue assumes in various states of motion, the range of positioning of nerves in various examples of aberrant anatomy.

As described further below, the process(or) 170 can include at least three (or more) functional modules (also termed processes/ors), including a motion process(or) 172 that determines motion changes and geometric variation within the subject tissue; a mapping process(or) 174 that builds models of the tissue and maps nerves (or other features of interest) in the tissue and correlates that map with respect to motion and geometry determined in the motion process(or); and vision system tools 176 that interoperate with acquired or synthetic image data of the tissue to locate and orient edges, features of interest, fiducials, etc. Note that these modules are illustrative of a wide range of possible organizations of functions and processes and are provided by way of non-limiting example.

Images of the surgical site can also be obtained via one or more imaging devices 190 that view the site 120 from an external and/or internal vantage point, and provide location and navigation information 192 (for example, via a surgical navigation system). This information can be provided to the computing device 150 and associated processor 170, as well as other processing devices that communicate with the system processor 170.

### II. Operational Procedure and Variations

In general, the system and method herein constructs a nerve location map, represented in a computational model, that is updated by obtaining the location of the stimulation of a nerve, together with classification of the physiological response (as measured by EMG, physical diameter or volumetric metrics, or other modalities) to that stimulation, so as to determine nerve proximity for a multiplicity of points in the surgical field. These points indicate to the user whether or not they are close to the subject nerve(s). The nerve location map is used to interpolate the probable path(s) of tissue innervation. For example, the system can cause identification of sampled points that have high proximity to a nerve, and linear interpolation is used to infer the proximity of intermediate points, between the sampled points, to the nerve. The system and method can also utilize information about typical anatomy (such as origin and destination of a nerve) to inform the nerve location map interpolation process. Thus, instead of performing a simple linear interpolation, if normal patient anatomy is such that the nerve is known to curve at a particular radius, the system and method can perform interpolation that includes a curve-fit of the endpoints to that anticipated curve, using methods such as least-mean-squares optimization.

In embodiments, the system and method can construct a nerve location map of the subject tissue/organ (as part of the feedback 160) by using motion/location information acquired from sensors built in to a surgical robotic system that provide direct measurement of the absolute location of the stimulating probe relative to a local coordinate system unique to the instrument, or a global coordinate system (e.g. a Cartesian system along three perpendicular axes plus rotations, such as the depicted global coordinate system 188, consisting of axes x, y and z and rotations θₓ, θ_{y} and θ_{z}) that is common to a plurality of instruments. Alternatively, or additionally, the system and method can construct a nerve location map by using location information acquired through a surgical navigation system (e.g. device 190), such as an electromagnetic or ceiling-mounted optical system, which measures the location of the stimulating probe in absolute (global) coordinates at the moment of stimulation, and fuses that information with information about the absolute location of the tissue, obtained by optical recognition of key tissue features, or by use of fiducial markers attached to the tissue that can be recognized by the surgical navigation system. It should be clear to those of skill that fiducial markers can be attached physical features, such as a surgical staple or clamp, may be features induced on the tissue itself, such as a laser-inscribed surgical tattoo, or may be features that are a natural part of the tissue, such as an easily-observed anatomical feature or spot discoloration, selected by the user (surgeon), or by the automated system for use as a motion-tracking marker.

The system and method can also construct the nerve location map in a manner that tracks nerve location relative to the tissue itself, in contrast to absolute coordinates referenced to the exterior of the body. By combining absolute location information about the (EMG) probe itself, together with location information about key anatomical features, the location of the nerve relative to the location of the tissue can be recorded/tracked. For example, in the case of prostate surgery, in which stimulation, pulse, etc. are causing motion of the tissues being analyzed, tracking tissue motion of a multiplicity of key reference points to convert absolute probe location information into tissue-relative location information is desirable.

In further embodiments, the nerve location map can be constructed using tissue-relative information achieved by direct observation (for example, by an endoscopic camera) of the location of the stimulating probe relative to recognized locations on the tissue of interest. Edges, textures, colors, topology, and other physical properties of the tissue can be utilized to track a multiplicity of locations on the tissue relative to the stimulating probe over time.

In embodiments, the nerve location map can consist of a 3-D model, such as a VOXEL representation, or can consist of a 2-D model such as a projection onto a plane, or a hybrid such as a surface topology model of the exposed portion of the tissue. Optionally the nerve location map can incorporate data representing the confidence with which a feature is known to be present. For example, in the case of hidden nerve detection, locations that are directly stimulated and produce muscle response have a high level of confidence, while points for which nerve presence is predicted via interpolation have a lower degree of confidence. The confidence can be stored as a score relative to a scale. Features with scores below a certain default or user-set threshold can be omitted from any system feedback.

As described above, the system and method can display (as part of the feedback 160 and/or VR or AR 162) the nerve location map to the user/surgeon in real-time, adjusted for actual tissue location. The display can include color-coding of the surgical field to form an augmented-reality viewpoint, or of other visual marking mechanisms such as drawing of a highlighted line along the estimated routing of the nerve on the endoscopic camera display output, and/or drawing of highlighted markers (such as a white ‘X’) at the points where nerve proximity was detected, or displaying a ‘heat map’ style representation showing the probability of the feature being in that location. In embodiments, the color of the markers, of the highlighted line, and of the surgical background can all be adjusted in a spatially-specific manner based upon the location map.

In further embodiments, the system and method can employ the above-described mapping and location mechanisms/procedures to detect and map vasculature locations—such as arteries, rather than nerve location. For example, fluid flow in arteries can be detected via (e.g.) Doppler ultrasound using an ultrasonic transducer mounted on the surgical probe. The ultrasonic transducer can be implemented as an emitter-only, with reception occurring at a different site, as a receiver array only, with transmission occurring at a different site, or the transducer can contain, or operate as, both an emitter and a receiver, performing echo/reflection-style talk-and-listen measurements.

In embodiments, the system and method can employ optical flow texture tracking techniques, such as the Kanade-Lucas-Tomasi (KLT) point feature tracking algorithm, to track motion of tissue within the surgical field, along with use of this tracking information to update the feature location map model. The map can be characterized in 2D (planar tracking with deformation), or full 3D, with 3D deformation estimated therein based upon surface motion observations. Alternatively, optical flow techniques, such as the above-referenced Tomasi-Kanade point feature tracking algorithm, can be used to track motion of a surgical probe within the surgical field. Use of this information to segment the probe versus the tissue, so as to update the model of tissue motion based exclusively on unobstructed tissue observations, thereby removing the potentially confounding effect of the probe. Additionally, tracking motion of the surgical probe may be used to further inform the location of the probe relative to the tissue for construction of the feature location map. By way of example, constrained Kalman filter techniques, such as those designed for vision-aided navigation, (e.g. the Multi-State Constraint Kalman Filter (MSCKF) algorithm) can be employed to enhance the performance of the above-referenced feature tracking algorithms based on estimates of the dynamic motion of the features being tracked. This can be especially helpful when the view of the feature being tracked becomes momentarily obscured by glare or by a surgical instrument. That is, the Kalman filter is employed to smooth the image data feed during tracking so as to omit events that are obscured by glare of tool-obstruction. Other appropriate smoothing or filtering algorithms/procedures can be employed in alternate embodiments in a manner clear to those of skill.

By way of further description, temporary obstructions to the imaged view, such as surgical instrument occlusion or glare can cause obscuration to features of interest that are tracked by the vision tools of the system. For example the tissue texture can be rendered in a particular color space, and the color features within the texture are tracked as the tissue moves (naturally or as a result of external stimulus). Glare, shadows and/or occlusion by a surgical instrument can cause some of the texture features to be obscured in certain states of tissue motion. Thus, in some image frames the feature is essentially lost to the tracking system. Illustratively, a model of the dynamics of the motion can used to estimate the missing data caused by the obscured feature. The model understand the general vectors of motion, undertaken by the tissue, and ascribes general rules to all adjacent points in the tissue. Thus, if all the other points moved to the right by (e.g.) 2 mm at a rate of 5 mm/sec, then it is very likely that the obscured point(s) are also moved by this amount, and their presence can be presumed in all images, despite obscuration in some.

In an embodiment the camera(s) that provide(s) observations can be moved to multiple locations in a controlled manner so as to provide multiple differently illuminated viewpoints of the features being tracked. Illustratively, multiple observations are acquired prior to stimulation of nerve-induced motion of the tissue. In an alternate embodiment, multiple observation sites (e.g. multiple cameras) are employed to obtain multiple observation perspectives of the same point(s) to be tracked. In embodiments, controlled motion and/or modulation of illumination sources can provide multiple views of the same point to be tracked. Motion of the illumination sources can be used to acquire image of the regions being tracked at a moment in time in which the illumination is configured in a manner that reduces glare induced by the lights. Also, motion of the illumination source(s) can be used to intentionally induce glare at a point of interest, so that any small motion of the tissue (such as induced by an instrument contacting the tissue) becomes apparent from the change in the intensity of the light reflected from that portion of the tissue surface. The detected motion can then be used to guide the operating limits of (e.g.) a surgical robot, providing a tactile indication to the user or providing a ‘stop here, contact has been achieved’ indication to an automated robot-arm controller (for example, via a force-feedback in the control stick).

In various embodiments, the system and method can track glare-highlighted motion (particularly overall motion direction) relative to non-glare motion as a vehicle for detecting motion of tissue. For instance, if the tissue is moving to the right and the glare is stationary or moving in another direction, this effect can provide queues that are used to separate the motion from the glare.

### III. Mapping Process

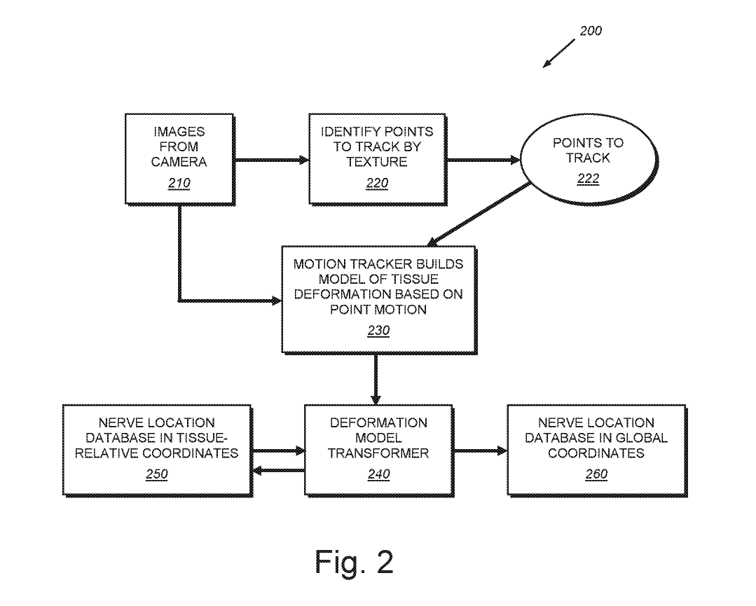

Having described the general operational considerations of the system and method and exemplary variations thereof, the following is a description of the mapping process in further detail. Reference is made to FIG. 2, which shows a flow diagram of a model-building process 200 according to an embodiment. As shown, by way of non-limiting example, images are provided in step 210. These images are typically acquired in real-time or near-real-time of the surgical site and associated tissue/organ. The images can also be provided from storage based upon a previous acquisition. The images are used by the vision system tools (176) to find points to track on the tissue (step 220). These points are stored (step 222) for use in the tracking step 230. Tracking can be based on contrast and/or color differences in the texture of the tissue, edges, vasculature, etc. Alternate techniques for tracking are also described above, including glare-based tracking, Doppler scans, medical imaging (e.g. MM), etc. Based on the located points, changes in the position of such points over a predetermined time interval are correlated with motion within the images (210) of the tissue/organ (which can be naturally occurring, or based upon applied stimulus from a (e.g. nerve stimulating) probe) by the tracking step. This tracked point motion establishes the general direction(s) in which tissue deforms, from which a deformation model can be constructed (step 240). The location of nerves in the tissue are established compared to the points, based upon detected nerve locations via the EMG response to localized nerve stimulation as well as other information available about known positions for nerves in the tissue (e.g. textbook locations) (step 250). This local positioning can be transformed by the model into a global coordinate system that can be relative to a robotic instrument or another reference (step 260). The information related to nerve location and nerve paths in various states of deformation is stored in a database (for example, tissue data 180).

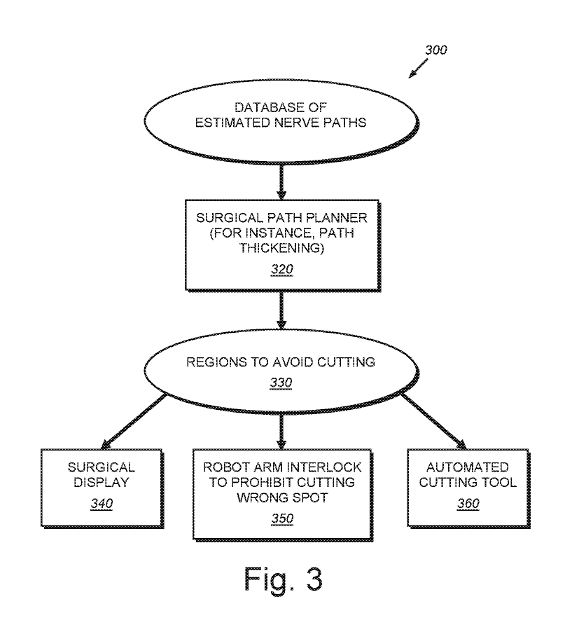

With reference to the procedure 300 of FIG. 3, the database of nerve paths 310 estimated by the procedure 200 are provided to a planner module in step 320. The planner can also employ various data from the tissue database 180 related to the properties of the tissue (such as the thickening of the nerve path in certain regions) to establish which regions of the tissue should be avoided (step 330). These regions can be expressed in an appropriate coordinate system. The results of the path planning step can then be provided to a variety of feedback devices. For example, they can be placed on a surgical display (step 340) that is projected to the user as a standalone display of the data, or a display that overlays the regions to avoid (potentially in colors) relative to a real-time image of the tissue. The regions can be morphed to follow the deformation of the organ as the vision system tools track motion in the organ as described above. The regions can also be overlaid onto a VR or AR display that is worn by the user. Alternatively (or additionally), the regions can be provided to a user-directed robotic surgical manipulator (step 350) using an appropriate coordinate system so that the manipulator locks out motion that would place a distally mounted tool in a no-go region of the tissue containing the nerve path. Similarly, the regions can be transmitted to an automated surgical tool and associated drive mechanism/controller to avoid incursion into no-go regions of the tissue (step 360).

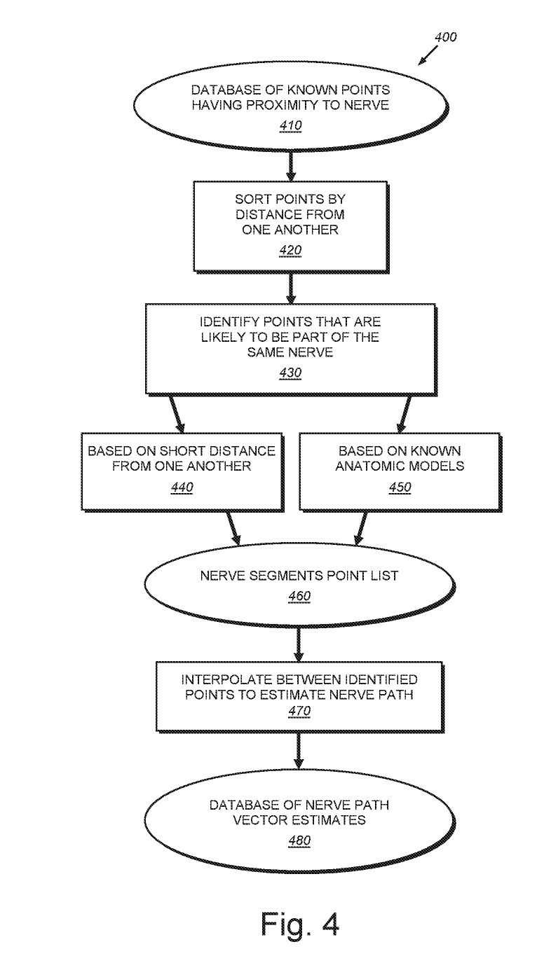

With reference to FIG. 4, a more detailed procedure 400 for determining a nerve path is shown. Points from the database determined above, which are known to be near a nerve are provided in step 410. These points are relative to a particular coordinate system and are thereby sorted based upon distance from each other in that coordinate system using (e.g.) known techniques in step 420. Points that are likely to be part of the same nerve are identified in step 430. This can be accomplished by line-fitting or curve fitting the points and confirming their fit. If the identified points exhibit a short distance from each other (step 440) and/or match known anatomic (textbook) models from the tissue data 180 (step 450), then they are placed in a nerve segment points list (step 460). After establishing a list of nerve segment points, the locations between these points that correspond to likely connecting segments can be established by interpolation or another known technique. This set of data is defined as the nerve path (step 470) and stored in the database (step 480).

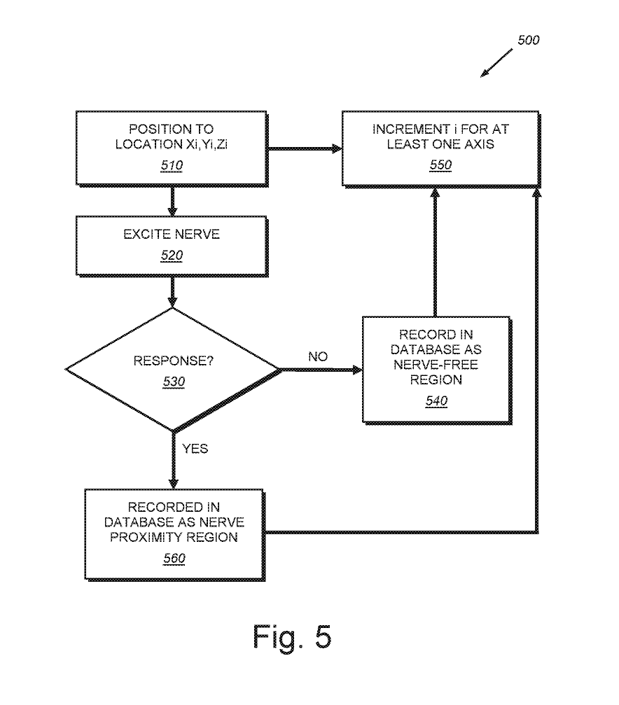

FIG. 5 details a basic, exemplary procedure for locating nerve-containing regions in a tissue during runtime operation. The probe (for example, a nerve stimulator), is moved to a relative (e.g. global) coordinate position Xi, Yi, Zi in the surgical field. The nerve is excited in step 520 and a response (for example, an EMG signal) is detected in decision step 530. If no response is observed, then this region is recorded in the point database as a nerve-free region (step 540) and the position (Xi, Yi, Zi) is adjusted for at least one of the coordinates Xi, Yi and Zi in step 550 and the procedure 500 repeats with step 510 at a new position i+1. The degree of granularity applied to this increment can vary depending upon the known density of nerves in the subject tissue, the overall size of the surgical field, and the sensitivity of the device, among other factors. If a response is detected (decision step 530), then the point is recorded in the database as being proximate to a nerve (step 560) and potentially part of a no-go region in the tissue. The procedure 500 branches to increment step 550 and repeats the excitation at a new location. The overall procedure 500 continues until a sufficient number of data points are acquired over the surgical field. Note that positioning can be performed by automated or manual control. In certain manual applications, the location of the probe can be established using a camera that correlates a location in space of the probe tip to a location on the imaged tissue. Likewise, an ultrasound imager can be used to localize the probe tip with respect to a coordinate system. Alternatively, a robotic manipulator can establish the location of the probe tip based upon internal (e.g. encoder) position information.

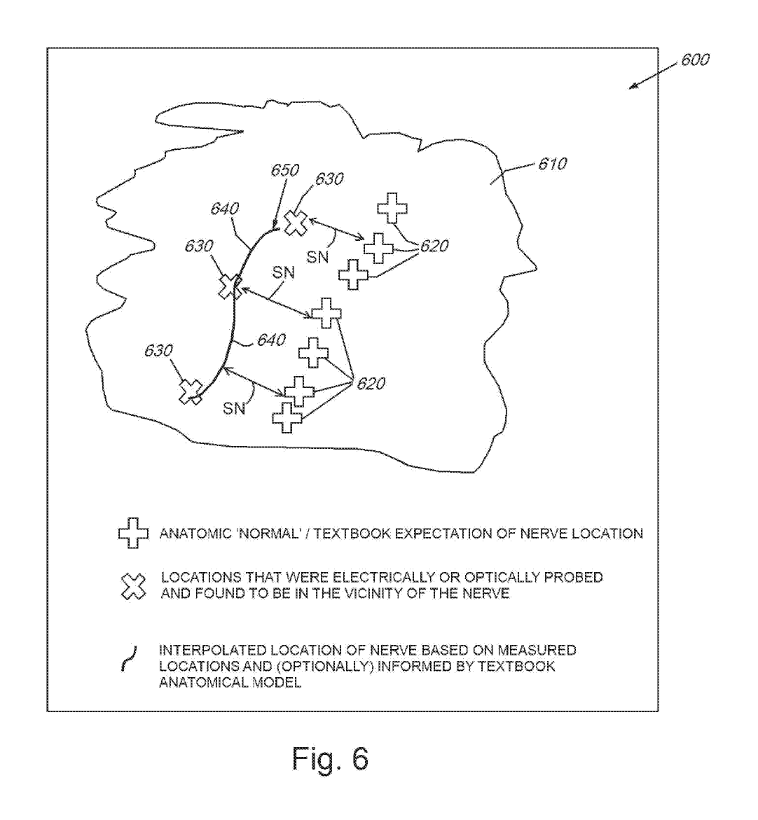

Reference is now made to FIG. 6 and representation 600, which shows an exemplary tissue/organ region 610 within a surgical field. The textbook expectation places the nerve path in the region of the crosses 620. However, through application of the procedures described above, actual points (Xs 630) were sensed via electrical (e.g. EMG) probing or optical (e.g. using vision tools) sensing. These points 630 are shown at a spacing SN from the expected path 620 that can prove critical to avoiding nerve damage in the event of a surgical procedure. This spacing SN can result from motion, deformation, aberrant anatomy, or a combination of such factors. As shown, path segments 640 are interpolated to fill in the complete, actual nerve path 650. The contour of the segments 640 generally matches that of the original path, as the interpolation has been guided by reference to a (conventional or standard) textbook path geometry, similar to that outlined by the crosses 620.

### IV. Operational Example

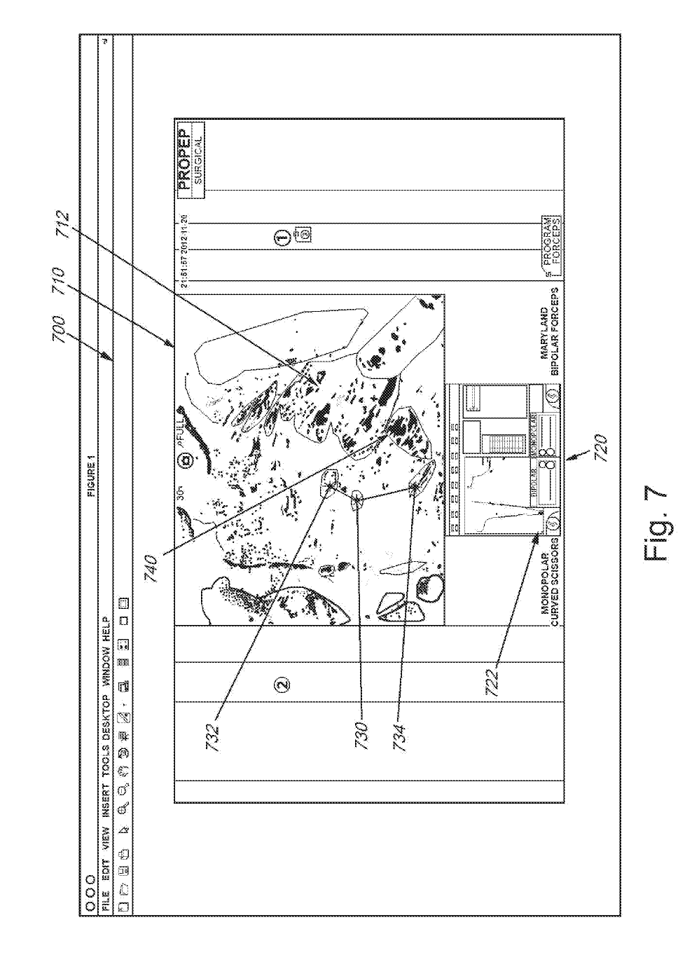

FIG. 7 shows an exemplary screen display (e.g. the GUI of a computing device, such as a tablet, laptop, PC, etc.) 700 that operates the system and method as described above—that is, the exemplary display can be part of the computing device 150 described in FIG. 1. The display 700 depicts one or more a real-time or stored image frames of a tissue region 710, and can be part of the above-described EMG implementation (e.g. the ProPep® system in which a stimulus by a probe is translated into a nerve response signal. The response is depicted graphically in the sub-display 720 along plot 722. The depicted tissue defines a texture that varies in color gradient (and/or shade), and includes both defined and soft edge features. These can be characterized by the associated vision system used herein to track motion as features of interest.

As shown, a probe 712, which can be any acceptable surgical instrument tip is shown engaging the tissue at a particular location. The probe in this example is mounted on the end of a surgical robot—such as the da Vinci® robotic surgical system available from Intuitive Surgical, Inc. of Sunnyvale, Calif. In the illustrative embodiment, the robot manipulator is modified to function as a unipolar device to operate as a nerve probe. As shown, the system has defined three points 730, 732 and 734 based on the response. Illustratively, features of interest, based on color gradient are used to localize the points. As shown, the image can contain glare (e.g. element 740) and occlusion from the probe) 712. These can be negated by use of an estimating process that can include a Kalman filtering process as described above. The points 730, 732 and 734 are shown displayed in overlay on the tissue, thereby allowing the surgeon to avoid them. By locating the stored features of interest during subsequent steps of the surgical procedure maintains these points relative to the tissue throughout a range of tissue and surgical instrument motion.

As part of the probe implementation, it can include circuitry to protect it as the nerve is excited. In general there is sufficient latency within nerve signal propagation to shut down the detection until the signal has propagated. Such circuitry can be implemented in a manner clear to those of skill in the art.

While the above-described example has focused upon locating and mapping regions of the tissue that contain a hidden anatomical feature (i.e. no go regions) or safe regions where it has been determined that are free of hidden features, there can exist unexplored regions that have not yet been probed by the system. These unexplored regions can be flagged in an image using an appropriate overlaid indicia (for example, a color tint, a surrounding graphical border, etc.), or lack of indicia. The regions, once probed are no longer marked as unexplored. In addition to displaying the unexplored regions to the user, who can then use a robot controller joystick or manual mechanism to probe them, the information related to such unexplored regions can be directed to an automated probe guidance system that can recommend to the user which locations to probe and/or automatically probes those locations. This effectively provides a hidden feature-finding autopilot for a robotic surgical system.

### V. Conclusion

It should be clear that the system and method herein provides an effective technique for locating actual nerve positions in a tissue or organ and retaining this information in a global coordinate system that can be associated with the tissue or organ in various states of deformation, motion and/or differing points of view. The location of nerve paths allows for manual and automated surgical procedure to be performed with a higher degree of safety and certainty that nerve (or other hidden anatomical structures) will not be disturbed/damaged.

It should also be clear that, while the above-described system and method uses hidden nerve detection by way of example, detection of other types of hidden structures can also be accomplished hereby. For example, instead of detecting a nerve via EMG, the user can detect an artery in the vicinity of the sampling probe through the use of ultrasound emitters and/or detectors located on the probe tip. Optionally, these emitters and/or detectors may interact with a second remote probe. For example, the emitter can be located in the probe while an array of listeners can be located at a predetermined remote distance therefrom. In general, the system and method effectively creates a spatial mapping of hidden features, updated in real-time based on sensor data, optionally augmented by pre-operative imaging data, and displayed to the surgeon or utilized by an automated surgical guidance system to enable protection or (in alternate embodiments) selective destruction of the hidden feature of interest.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention For example, also as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor here herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A system for spatial mapping of hidden features in a tissue comprising:
a source of sensed point data established with respect to a coordinate system established relative to a region of the tissue containing the hidden features;
a mapping module that builds data related to hidden feature locations in the coordinate system based upon at least one of (a) sensed motion in the tissue, (b) stored anatomical models, and (c) stored information related to the tissue; and
a data storage module that stores the hidden feature locations to either locate or avoid the hidden features during a surgical procedure.

2. The system as set forth in claim 1, wherein the hidden features comprise one or more nerve paths in the tissue.

3. The system as set forth in claim 2, wherein the sensed point data is determined using a nerve stimulator in combination with a sensor that measures response to stimulation.

4. The system as set forth in claim 3, wherein the mapping module includes an interpolation process that fills in nerve path regions between the sensed point data.

5. The system as set forth in claim 4, wherein the mapping module is, at least one of, (a) updated in real-time based on sensor data and (b) augmented by pre-operative imaging data of the tissue.

6. The system as set forth in claim 5, wherein the nerve path is displayed to a user and/or utilized by an automated surgical guidance system.

7. The system as set forth in claim 6, wherein the display includes at least one of a VR and AR display.

8. The system as set forth in claim 1, wherein the mapping module is provided with images of the tissue in a plurality of positions based upon motion, the images including identified features of interest, and wherein the mapping module includes an estimation process that is arranged to estimate locations of the features of interest in each of the plurality of positions, including a position in which one or more of the features of interest are temporarily blocked from view by an obscuration.

9. The system as set forth in claim 1, wherein the features of interest are established in the texture of the tissue based upon variations in at least one of color, edges and grayscale level, and the obscuration comprises at least one of glare, shadow and instrument occlusion.

10. The system as set forth in claim 9, wherein the estimation process employs a stored model of the dynamics of the motion to determine a location of the one or more temporarily blocked features of interest based upon locations of the one or more features of interest when unblocked from view.

11. The system as set forth in claim 10, wherein the estimation process includes a Kalman filter process.

12. The system as set forth in claim 1, wherein the hidden feature locations define a multi-dimensional map of hidden feature locations, the multi-dimensional map being incorporated into the coordinate system relative to the region of tissue, whereby multiple sensed data points can be observed simultaneously relative to the tissue.

13. A method for spatial mapping of hidden features in a tissue comprising the steps of:
establishing sensed point data with respect to a coordinate system relative to a region of tissue containing the hidden features;
building data related to hidden feature locations in the coordinate system based upon at least one of (a) sensed motion in the tissue, (b) stored anatomical models, and (c) stored information related to the tissue; and
storing the hidden feature locations to either locate or avoid the hidden features during a surgical procedure.

14. The method as set forth in claim 13, wherein the hidden features comprise one or more nerve paths in the tissue.

15. The method as set forth in claim 14, further comprising, determining the sensed point data using a nerve stimulator in combination with a sensor that measures response to stimulation.

16. The method as set forth in claim 15, further comprising, augmenting the data related to the hidden features based upon pre-operative imaging data of the tissue.

17. The method as set forth in claim 16, further comprising, displaying the nerve path to a user and/or utilizing of the nerve path by an automated surgical guidance system.

18. The method as set forth in claim 13, further comprising, providing images of the tissue in a plurality of positions based upon motion, the images including identified features of interest, and estimating locations of the features of interest in each of the plurality of positions, including a position in which one or more of the features of interest are temporarily blocked from view by an obscuration.

19. The method as set forth in claim 18, further comprising, establishing the features of interest in the texture of the tissue based upon variations in at least one of color, edges and grayscale level, and the obscuration comprises at least one of glare, shadow and instrument occlusion.

20. The method as set forth in claim 19, further comprising, employing a stored model of the dynamics of the motion to determine a location of the one or more temporarily blocked features of interest based upon locations of the one or more features of interest when unblocked from view.

21. The method as set forth in claim 20, further comprising, employing a Kalman filter process.

22. The method as set forth in claim 13 wherein the step of storing includes determining and mapping one or more unexplored regions of the tissue that are free of sensed point data and providing indications of the one or more unexplored regions to the user.

23. The method as set forth in claim 22, wherein the unexplored regions are provided to a robotic sensing arrangement to guide further sensing operations in the unexplored regions.

24. The method as set forth in claim 23, wherein the step of building comprises compiling data related to the hidden feature locations into a multi-dimensional map of hidden feature locations, the multi-dimensional map being incorporated into the coordinate system relative to the region of tissue, and providing multiple sensed data points to be observed simultaneously relative to the tissue.
